(19) United States
(12) Patent Application Publication
(10) Pub. No.: US 2025/0355069 A1
(43) Pub. Date: Nov. 20, 2025

(54) Computer-Implemented Method for Operating a Magnetic Resonance Device, Magnetic Resonance Device, Computer Program and Electronically Readable Data Medium

(71) Applicant: Siemens Healthineers AG, Forchheim (DE)

(72) Inventors: Christian Meixner, Erlangen (DE); Peter Schmitt, Weisendorf (DE); Max Müller, Erlangen (DE)

(73) Assignee: Siemens Healthineers AG, Forchheim (DE)

(21) Appl. No.: 19/209,976

(22) Filed: May 16, 2025

(30) Foreign Application Priority Data
May 17, 2024 (DE) 10 2024 204 626.4

(51) Int. Cl. G01R 33/483; G01R 33/54; G01R 33/563

(52) U.S. Cl. CPC G01R 33/4838; G01R 33/4835; G01R 33/543; G01R 33/5635

(57) ABSTRACT

A computer-implemented method for operating a magnetic resonance device during the acquisition of magnetic resonance data from an examination region of an examination object may use a magnetic resonance sequence. The MR sequence may include a saturation section in which, by means of a saturation radiofrequency pulse which is generated when a saturation gradient pulse is applied, a saturation with respect to a spin species that is to be imaged is performed in at least one first saturation region. The saturation radiofrequency pulse may be designed as a multiband pulse by means of which, in addition, in at least one second saturation region, the magnetization with respect to a second spin species that is not to be captured is saturated.

### CROSS REFERENCE TO RELATED APPLICATIONS

This patent application claims priority to German Patent Application No. 102024204626.4, filed May 17, 2024, which is incorporated herein by reference in its entirety.

### BACKGROUND

### Field

The disclosure relates to a computer-implemented method for operating a magnetic resonance device during the acquisition of magnetic resonance data from an examination region of an examination object, wherein a magnetic resonance sequence is used comprising a saturation section in which, by means of a saturation radiofrequency pulse which is generated when a saturation gradient pulse is applied, a saturation with respect to a spin species that is to be imaged is performed in at least one first saturation region. The disclosure additionally relates to a magnetic resonance device, a computer program and an electronically readable data medium.

### Related Art

Conventionally, imaging modalities are known in which saturation radiofrequency pulses can also relate to the spin species actually to be imaged, i.e. forming the target of the imaging, typically spins of protons bound in water. Angiographic imaging procedures, in particular in time-of-flight angiography (TOF angiography), are one example of this. In that context it is known to output locally acting saturation pulses with respect to the spin species that is to be imaged in order to achieve a venous saturation, i.e. to mask out blood in the veins of the examination object, in particular a patient. In the case of angiography of the head of a patient it is known for example to saturate an outermost transverse slice of the head comprising the sinuses, and consequently the blood flowing into the sinus.

On the other hand, it is desirable in many magnetic resonance imaging tasks to suppress magnetic resonance signals of at least one other spin species that is not the target of the image acquisition. Typically, in this case, when the spin species to be captured are water spins, this concerns fat spins, i.e. protons bound in fat. The signal of the other spin species can lead to image artifacts. In the already mentioned TOF angiography, for example, vessels in certain regions can be overlaid by fat signal. Often, the bright fat signal is also disruptive in the frequently employed visualization of a three-dimensional magnetic resonance dataset as a “maximum intensity projection” (MIP). For example, when the head is the examination region, intracranial vessels can be seriously impacted in terms of their visibility by extracranial fat.

In magnetic resonance sequences which already use an above-described regional saturation of the first spin species forming the target of the imaging, in particular of water spins, and often have an upper limit in terms of repetition time (TR), fat saturation techniques, which additionally cost time, can be used only to a very limited extent. Such conventional fat saturation techniques include the use of spectral fat saturation pulses as well as inversion pulses (STIR and SPAIR). With spectral fat saturation pulses, there is furthermore the problem that an unwanted presaturation of the actually wanted magnetic resonance signal of the first spin species, in angiography of the head, for example, of the blood signal in thorax or neck, can occur which leads to a reduction in image quality.

In an article by Sebastian Schmitter et al., “Contrast Enhancement in TOF Cerebral Angiography at 7 T Using Saturation and MT Pulses Under SAR Constraints: Impact of VERSE and Sparse Pulses”, Magnetic Resonance in Medicine 68 (2012), pages 188-197, it is observed that VERSE pulses at off-resonances, as occur for example due to the chemical frequency shift between spin species, exhibit a strongly shifted excitation profile. At a main magnetic field strength of 7 T, the frequency shift between water spins and fat spins lies at around 1000 Hz. The slice excited by the corresponding shifted portion of the excitation profile of the VERSE pulse lies far away from the excitation profile at a frequency shift of 0 Hz and can therefore be used for simultaneous fat saturation. However, this is not possible at lower magnetic field strengths of the main magnetic field since, for example at 3 T, the frequency shift lies at approx. 300 Hz and consequently very close to the excitation profile of the regional saturation, in particular venous saturation, at a frequency shift of 0 Hz. Since the frequency shift between water spins and fat spins drops further linearly at lower magnetic field strengths, this relatively randomly occurring effect is likewise unusable.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 is a flowchart of a method according to one or more exemplary embodiments of the disclosure.

FIG. 2 shows a sequence diagram of a magnetic resonance sequence according to one or more exemplary embodiments that is usable for the method according to the disclosure.

FIG. 3 illustrates a magnetic resonance device according to one or more exemplary embodiments of the disclosure.

FIG. 4 illustrates a controller of the magnetic resonance device one or more exemplary embodiments of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the disclosure is to provide a time-efficient means of using a regional saturation of a first spin species that is to be imaged and an, in particular likewise regional, saturation of a second spin species, in particular at lower magnetic field strengths of the main magnetic field.

In a method of the type cited in the introduction, it is provided according to the disclosure that the saturation radiofrequency pulse is designed as a multiband pulse by means of which, in addition, in at least a second saturation region, the magnetization with respect to a second spin species that is not to be captured is saturated.

A magnetic resonance sequence comprising a saturation section is used. Further possible sections of the magnetic resonance sequence comprise, as is generally known, an excitation section having at least one excitation radiofrequency pulse, as well as a readout section for measuring the magnetic resonance data. Furthermore, the magnetic resonance sequence may also comprise a preparation section, for example in the case of diffusion sequences and the like.

In this process, a saturation with respect to the second spin species that is not to be captured may be performed in a second saturation region corresponding to an excitation region of the excitation section or comprising the same. If the saturation regions are slices, a second saturation slice of the second saturation region of the excitation slice can correspond to or comprise the excitation slice. If the magnetization of the second spin species is saturated in the excitation region, it is no longer affected by the excitation pulse or makes no contribution to the magnetic resonance signal.

Now, in the saturation section, as is known for local saturation processes, for example venous saturation in TOF angiography, a saturation radiofrequency pulse is output by means of a radiofrequency coil array of the magnetic resonance device. In parallel therewith, at least one saturation gradient pulse is output in order to permit a spatial selection, in particular a slice selection. It is proposed to design the saturation radiofrequency pulse as a multiband pulse by means of which a regional saturation with respect to the first spin species and a likewise regional saturation with respect to the second spin species can be achieved simultaneously. In this way the repetition time can be shortened, in particular in time-critical acquisition processes, which leads to an improvement in image contrast, in particular, therefore, also in the signal-to-noise ratio and consequently in image quality. Furthermore, the overall acquisition time is reduced.

In this case the saturation regions are beneficially spaced apart from one another or disjoint. That means the subpulses of the multiband pulse may act on spatially separated saturation regions, in particular saturation slices. Whereas conventional multiband pulses are used to excite multiple slices or regions, the saturation radiofrequency pulse designed as a multiband pulse serves for the simultaneous saturation of different spin species in multiple saturation regions.

In particular, it can be provided as a frequently employed example that the first spin species comprises spins of protons bound in water (water spins) and/or the second spin species comprises spins of protons bound in fat (fat spins). Then, both a regional saturation for the water spins as first spin species and a fat saturation can therefore be achieved in a single saturation radiofrequency pulse.

A beneficial application area is given when the magnetic resonance sequence serves for angiographic imaging, in particular for TOF angiography, and a venous saturation is performed in the first saturation region. In that case a venous saturation and a fat saturation can therefore be achieved simultaneously with the aid of a single saturation radiofrequency pulse. The examination region may in this case be the head of a patient as examination object, for example, although the method can of course also be applied generally to other examination regions. In an acquisition of magnetic resonance data in TOF angiography of the head of a patient, at least one of the at least one first saturation region can comprise a region of the cranial sinuses, in particular an uppermost transverse slice as a saturation slice. For the second saturation region, regions of the head in which fat is present, in particular a region of the eyes, can then be used. In this way venous fractions of the blood are presaturated and image artifacts due to fat are avoided simultaneously.

A multiband pulse may generally be understood as a superposition of two subpulses, such as described for example in an article by Markus Barth et al., “Simultaneous Multislice (SMS) Imaging Techniques”, Magnetic Resonance in Medicine 75 (2016), pages 63 to 81. A slice-selective, complex radiofrequency pulse which can form a subpulse can be described in functionally mathematical terms in its evolution as

$$R F ( t ) = A ( t ) \cdot P ( t ) ,$$

where A(t), as the pulse shape function, describes the complex radiofrequency waveform which determines the slice profile in conjunction with the slice-selective gradient, and P(t), as an additional phase modulation function, describes the slice position (Δω) and its phase (φ) at TE=0,

$$P ( t ) = {e}^{i Δω t + φ} .$$

Here, the slice position is described by a slice frequency shift Δω relative to the nominal Larmor frequency present due to the magnetic field gradient generated by the gradient pulse. In order to obtain an effect on multiple slices using the same pulse shape function (which for example can describe a SINC pulse or a hyperbolic secant pulse), multiple phase modulation functions, each assignable to a subpulse, are added together. This approach can also be applied to the design of the present saturation radiofrequency pulse, which in an embodiment for N saturation regions can have the functional form

$$R {F}_{M B} ( t ) = A ( t ) \cdot {\sum }_{N} {e}^{i {Δω}_{n} t + φ} .$$

In practice, an advantageous development of the present disclosure can provide that at least one slice selection of the multiband pulse for each saturation region is described by a frequency shift value of a phase modulation function, wherein the frequency shift value of the phase modulation function is chosen
for each first saturation region as a slice frequency shift which is chosen as a function of the magnetic field gradient that is provided by the gradient pulse for the definition of a second saturation slice forming or containing the first saturation region, and
for each second saturation region as the sum of a slice frequency shift which is chosen as a function of the magnetic field gradient that is provided by the gradient pulse for the definition of a second saturation slice forming or containing the second saturation region and the chemical frequency shift between the first and the second spin species.

Expressed in formulae, therefore, the following can be written for the case of precisely two saturation regions, namely a first saturation region and a second saturation region, for the phase modulation functions of the subpulses (TP):

$${P}_{TP 1} ( t ) = {e}^{i {Δω}_{S 1} t + φ} and$$

$${P}_{TP 2} ( t ) = {e}^{i ( Δ {ω}_{S 2} + Δ {ω}_{c V} ) t + φ}$$

Here, Δω_{S1} denotes the slice frequency shift for the first saturation slice of the first saturation region based on the magnetic field gradient generated by the saturation gradient pulse, i.e. it describes the slice position of the first saturation slice in which the regional saturation of the first spin species, for example the venous saturation, is to take place. Analogously, Δω_{S2} describes the slice position of the second saturation slice of the second saturation region in which the fat saturation is to take place. The second saturation slice corresponds particularly advantageously at least to some extent to the excitation slice which is excited in an excitation section of the magnetic resonance sequence. With regard to the second subpulse for the second saturation region, however, there is the added issue that the second spin species, not the first species, is to be affected there, for which reason the chemical frequency shift (often also referred to as off-resonance) between the first spin species and the second spin species is also included with Δω_{cV} in the total frequency shift value Δω₂. The chemical frequency shift between protons bound in fat and protons bound in water amounts to approx. 3.5 ppm, i.e. roughly 220 Hz at a magnetic field strength of the main magnetic field of 1.5 T, for example.

The thickness of the first saturation slice and the second saturation slice is determined in this case via the simultaneously played-out saturation gradient pulse, in particular its amplitude, together with the frequency bandwidth of the respective subpulse.

It should also be noted at this point that in many cases the magnetic field gradient, in particular its direction, can skillfully be chosen such that a slice selected with the total frequency shift value Δω₂ as the slice frequency shift lies far enough away to avoid an unwanted saturation of the first spin species in a relevant region.

As already explained, the mathematical description of the evolution of the multiband pulse for each saturation region can comprise a pulse shape function describing the pulse shape of the respective fraction that was denoted by A(t) in the above formulae. In this case, on the one hand, as described by way of example above, the pulse shape function can be chosen as the same for all saturation regions. However, it is also conceivable in beneficial developments that for at least one saturation region the pulse shape function is chosen as different from at least one other saturation region in order to adjust the slice thickness of the \respective saturation slice and/or of the slice profile of the respective saturation slice. For example, the thicknesses of the respective saturation slices can be adjusted by way of different bandwidths. An implementation of different slice profiles is also conceivable by way of the pulse shape function. A multiband pulse having different pulse shape functions for the subpulses can be described as

$$R {F}_{M B} ( t ) = {\sum }_{N} {A}_{n} ( t ) \cdot {e}^{i {Δω}_{n} t + φ} .$$

In this case it should also be noted at this point that the present disclosure also permits a multiband pulse to be defined and used for multiple first saturation regions and/or multiple second saturation regions. In this way it is therefore possible for example to realize a regional saturation of the magnetization of the first spin species at multiple points in the examination object, for example at multiple venous confluence points or confluence regions. Furthermore, it is also possible in addition or alternatively to achieve a saturation with respect to the second spin species, for example a fat saturation, in multiple second saturation regions, in particular therefore in multiple second saturation slices. This can be combined for example with excitation radiofrequency pulses in excitation sections which likewise relate to multiple excitation slices. A high degree of flexibility is therefore present in this regard.

In a beneficial embodiment it can be provided that the multiband pulse is designed as a VERSE pulse. VERSE (variable-rate selective excitation) pulses use a time-varying gradient in order to adjust the radiofrequency pulse shape in such a way that the specific absorption rate (SAR) can be significantly reduced. It can therefore be achieved in this way that not only can the two spin species be saturated simultaneously in corresponding saturation regions but a reduction in the exposure of the examination object, in particular a patient, can also be achieved. An implementation of multiband VERSE pulses has already been demonstrated for excitation radiofrequency pulses in the prior art, so an implementation as a saturation radiofrequency pulse is also possible.

The method is in this case may be employed at lower magnetic field strengths of the main magnetic field. It can therefore be provided that the magnetic resonance device generates a main magnetic field having a magnetic field strength of 3 T or less. In principle, however, its use at other magnetic field strengths is also conceivable.

In addition to the method, the present disclosure also relates to a magnetic resonance device comprising a main magnet unit having a main magnet for generating a main magnetic field, a gradient coil array for generating gradient pulses, a radiofrequency coil array for generating radiofrequency pulses, and a control device comprising a sequence unit for controlling the acquisition of magnetic resonance data, the sequence unit being embodied for acquiring image data from an examination region of an examination object by means of a magnetic resonance sequence having a saturation section in which, by means of a saturation radiofrequency pulse designed as a multiband pulse which is generated when a gradient pulse is applied, both a saturation with respect to a spin species that is to be acquired in at least one first saturation region is performed and, in addition, in at least one second saturation region, the magnetization with respect to a second spin species that is not to be captured is saturated. In other words, the control device is embodied for performing the method according to the disclosure. All statements made with respect to the method according to the disclosure can be applied analogously to the magnetic resonance device according to the disclosure, and vice versa, such that the already cited advantages can also be obtained with the magnetic resonance device.

The control device (controller) may comprise at least one processor and at least one storage means. Functional units can be formed by means of hardware and/or software in order to perform steps of the method according to the disclosure. Whereas sequence units for performing magnetic resonance sequences, in particular by driving the gradient coil array and the radiofrequency coil array of the magnetic resonance device, may already be generally known, the sequence unit in the present example may be configured to use a multiband pulse as the saturation radiofrequency pulse for the regional saturation with respect to the first spin species in a first saturation region and for the saturation with respect to the second spin species in a second saturation region. Depending on the actual imaging task, a sequence determination unit may also be present in order to adapt a basic form of the magnetic resonance sequence to fit the specific circumstances of the imaging task, for example, to choose frequency shift values and/or bandwidths and slice profiles as appropriate.

A computer program according to the disclosure can be loaded directly into a storage means of a control device of a magnetic resonance device and comprises program means which are configured such that when the computer program is executed on the control device, the latter is caused to perform the steps of a method according to the disclosure. The computer program can be stored on an electronically readable data medium according to the disclosure, which therefore comprises control information stored thereon which includes at least one computer program according to the disclosure and is configured such that when the data medium is used in a control device of a magnetic resonance device, said control device being configured to perform a method according to the disclosure. The data medium may be a non-transitory data medium, for example, a CD-ROM.

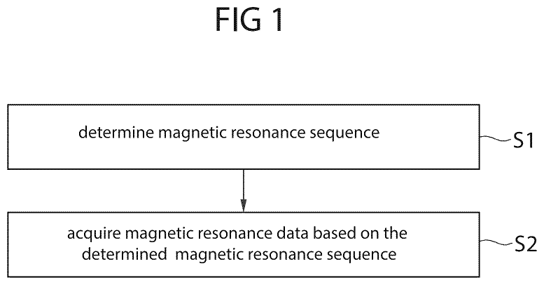

FIG. 1 shows a flowchart of an exemplary embodiment of the method according to the disclosure in which magnetic resonance data is to be acquired in the course of a TOF angiography scan in a head as the examination region of a patient as the examination object. A magnetic resonance device is used, the main magnetic field of which can have a magnetic field strength of 3 T or 1.5 T, for example.

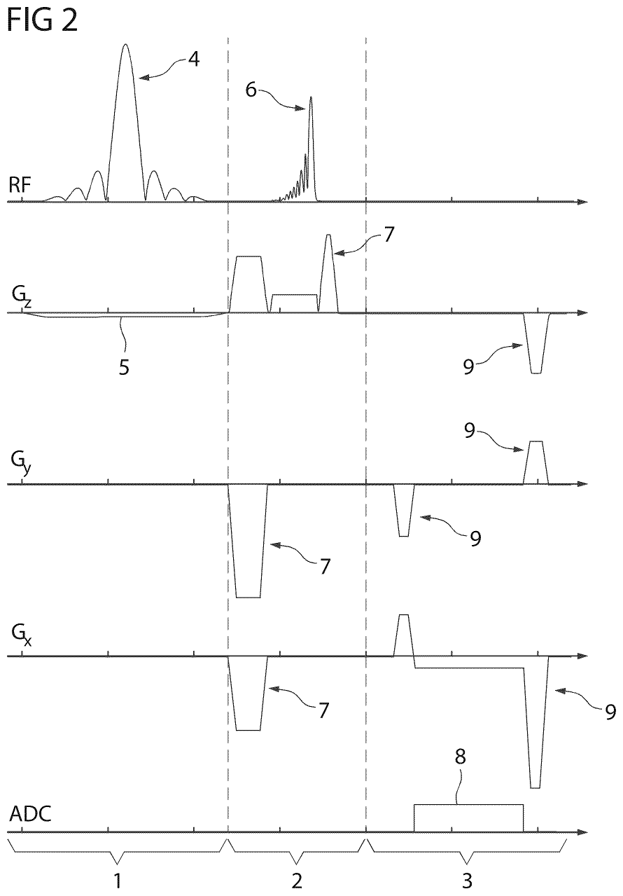

In this case, in a step S1, the magnetic resonance sequence that is to be used is determined, in the present example by suitable parameterization of a template for the sequence workflow. An example of such a magnetic resonance sequence is shown in FIG. 2, wherein in the present case only the portions relevant to the present disclosure are to be described in detail.

The magnetic resonance sequence comprises a saturation section 1, an excitation section 2 and a readout section 3. The saturation section 1 in this case comprises a saturation radiofrequency pulse 4 and a saturation gradient pulse 5, in the present example in the z-direction as the slice selection direction, which is output simultaneously with the saturation radiofrequency pulse 4.

An excitation radiofrequency pulse 6, accompanied by corresponding gradient pulses 7 in the excitation section 2, is used in order to excite unsaturated spins in an excitation region, in the present example an excitation slice, such that the decay of said excitation can be read out as a magnetic resonance signal in the readout section 3 in a corresponding readout time window 8 using further gradient pulses 9.

In this case the saturation radiofrequency pulse 4 is a multiband pulse which in the present example comprises two superposed subpulses. A first subpulse relates in this case to the saturation of water spins as the first spin species, which is the target of the acquisition, in a first saturation region, here a first saturation slice lying outside of the excitation region. In practice it serves for the venous saturation and can relate for example to the cranial sinuses in order to presaturate the water spins of the venous blood before these reach the corresponding veins of the excitation region for the purpose of excitation. The frequency shift value Δω₁ of the phase modulation function of the first subpulse is therefore chosen as the slice frequency shift Δω_{S1} of the corresponding first saturation slice according to the magnetic field gradient generated by the gradient pulse 5.

The second subpulse relates to the saturation of fat spins as the second spin species (fat saturation) in a second saturation region, here a second saturation slice corresponding to the excitation slice. The frequency shift value Δω₂ of the phase modulation function of the second subpulse is therefore yielded as a sum which comprises, as the first summand, the slice frequency shift Δω_{S2} of the corresponding second saturation slice according to the magnetic field gradient generated by the gradient pulse 5 and, as the second summand, the chemical frequency shift Δω_{cV} of the magnetic resonance frequencies between protons bound in water and protons bound in fat in order to relate to the correct second spin species, fat spins, i.e.

$$Δ {ω}_{2} = Δ {ω}_{S 2} + Δ {ω}_{c V} .$$

These frequency shift values Δωₙ are set in step S1 for the actual imaging task in addition to other parameters of the magnetic resonance sequence. In the present example, the pulse shape, described by a corresponding pulse shape function, is the same for both subpulses. However, it is also conceivable to choose these as different, for example when different slice thicknesses and/or slice profiles are to be used for the first and second saturation slices. Furthermore, multiple first subpulses may also be used for multiple first saturation regions and/or multiple second subpulses for multiple second saturation regions.

Even though a SINC pulse shape is indicated by way of example in FIG. 2, it is particularly advantageous to use a VERSE pulse as the multiband pulse since then it is possible to obtain a low SAR in addition to achieving two saturation targets with one saturation radiofrequency pulse 4.

In a step S2 of FIG. 1, the magnetic resonance sequence determined according to step S1 is then used in order to acquire magnetic resonance data. Thanks to the two saturation targets reached, a good image quality is achieved. Short repetition times and overall acquisition times are possible as a result of using the common saturation radiofrequency pulse 4.

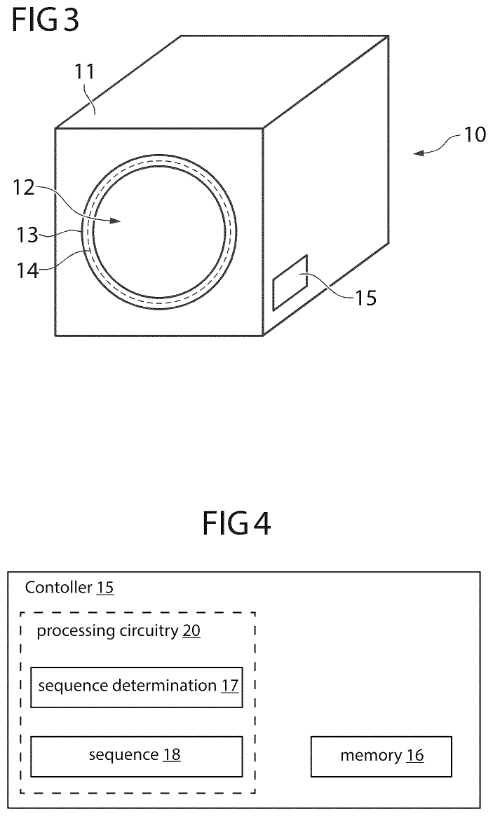

FIG. 3 shows a schematic diagram of a magnetic resonance device 10 according to the disclosure. This comprises, as is generally known, a main magnet unit 11 having a main magnet (not shown in more detail here). The main magnet generates a main magnetic field having a magnetic field strength of, for example, 3 T or less. A patient can be introduced into a patient bore 12 of the main magnet unit 11 by means of a patient couch (not shown in more detail here) for the imaging procedure. A gradient coil array 13 and a radiofrequency coil array 14 are indicated surrounding the patient bore 12. In addition to the radiofrequency coil array 14, a local coil array, in the case of imaging of the head, for example, a head coil, can be used as a further radiofrequency coil array. The operation of the magnetic resonance device 10 is controlled by a control device (controller) 15, the functional layout of which is explained in more detail with regard to the relevant components in FIG. 4.

As shown in FIG. 4, the controller 15 may comprise a storage means, such as memory 16 in which information, for example, acquired magnetic resonance data and/or parameterized magnetic resonance sequences, can be stored. The controller 15 may further comprise processing circuitry 20 that may be configured to perform one or more functions and/or operations of the controller 15. The processing circuitry 20 may comprise a sequence determination unit (sequence determiner) 17 and a sequence unit (sequencer) 18. Magnetic resonance sequences for imaging tasks may be collected in the sequence determination unit 17 and adapted to fit the corresponding imaging task, in particular comprising the parameterization of the multiband pulse according to step S1. Magnetic resonance data can then be acquired accordingly in a sequence unit 18 using the magnetic resonance sequences, in particular also according to step S2.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method for operating a magnetic resonance device during the acquisition of magnetic resonance data from an examination region of an examination object, the method comprising:
determining a magnetic resonance sequence comprising a saturation section including a saturation radiofrequency pulse generated in response to application of a saturation gradient pulse, a saturation with respect to a spin species that is to be imaged being performed in at least one first saturation region, wherein the saturation radiofrequency pulse is configured as a multiband pulse adapted to saturate magnetization with respect to a second spin species that is not to be captured; and
acquiring the magnetic resonance data based on the determined magnetic resonance sequence.

2. The method as claimed in claim 1, wherein the first spin species comprises spins of protons bound in water and/or the second spin species comprises spins of protons bound in fat.

3. The method as claimed in claim 1, wherein the first spin species comprises spins of protons bound in water and the second spin species comprises spins of protons bound in fat.

4. The method as claimed in claim 1, wherein at least one slice selection of the multiband pulse for each saturation region is described by a frequency shift value of a phase modulation function, wherein the frequency shift value of the phase modulation function is selected for each:
first saturation region as a slice frequency shift chosen as a function of the magnetic field gradient provided by the saturation gradient pulse to define a second saturation slice forming or containing the first saturation region, and
second saturation region as a sum of a slice frequency shift chosen as a function of the magnetic field gradient provided by the saturation gradient pulse to define a second saturation slice forming or containing the second saturation region and a chemical frequency shift between the first and the second spin species.

5. The method as claimed in claim 1, wherein the multiband pulse for each saturation region comprises a pulse shape function describing a pulse shape of the respective portion.

6. The method as claimed in claim 5, wherein:
the pulse shape function is selected to be the same for all saturation regions; or
for at least one saturation region, the pulse shape function is selected to be different from at least one other saturation region to adjust a slice thickness of the respective saturation slice and/or a slice profile of the respective saturation slice.

7. The method as claimed in claim 5, wherein the pulse shape function is selected to be the same for all saturation regions.

8. The method as claimed in claim 5, wherein, for at least one saturation region, the pulse shape function is selected to be different from at least one other saturation region to adjust a slice thickness of the respective saturation slice and/or a slice profile of the respective saturation slice.

9. The method as claimed in claim 1, wherein the multiband pulse is designed as a variable-rate selective excitation (VERSE) pulse.

10. The method as claimed in claim 1, wherein: (a) the magnetic resonance device generates a main magnetic field having a magnetic field strength of 3 T or less; and/or (b) the magnetic resonance sequence serves for angiographic imaging, a venous saturation being performed in the first saturation region.

11. The method as claimed in claim 1, wherein: (a) the magnetic resonance device generates a main magnetic field having a magnetic field strength of 3 T or less; and (b) the magnetic resonance sequence serves for angiographic imaging, a venous saturation being performed in the first saturation region.

12. One or more non-transitory media storing instructions that, when executed by one or more processors, cause the one or more processors to perform the method of claim 1.

13. An apparatus comprising:
one or more processors; and
memory storing instructions that, when executed by the one or more processors, cause the apparatus to:
determine a magnetic resonance sequence comprising a saturation section including a saturation radiofrequency pulse generated in response to application of a saturation gradient pulse, a saturation with respect to a spin species that is to be imaged being performed in at least one first saturation region, wherein the saturation radiofrequency pulse is configured as a multiband pulse adapted to saturate magnetization with respect to a second spin species that is not to be captured; and
control a magnetic resonance device to acquire magnetic resonance data based on the determined magnetic resonance sequence.

14. A magnetic resonance device comprising:
a main magnet unit including a main magnet configured to generate a main magnetic field;
a gradient coil array configured to generate gradient pulses;
a radiofrequency coil array configured to generate radiofrequency pulses; and
a controller configured to control an acquisition of magnetic resonance data and acquire image data from an examination region of an examination object based on a magnetic resonance sequence,
wherein the magnetic resonance sequence comprises a saturation section in which, by a saturation radiofrequency pulse configured as a multiband pulse which is generated in response to an application of a saturation gradient pulse, both a saturation with respect to a spin species that is to be imaged in at least one first saturation region is performed, and, in at least one second saturation region, magnetization with respect to a second spin species that is not to be captured is saturated.

15. The magnetic resonance device as claimed in claim 14, wherein the controller comprises a sequence unit configured to control the acquisition of the magnetic resonance data and acquire the image data.
